(19) United States
(12) Patent Application Publication
(10) Pub. No.: US 2008/0274537 A1
(43) Pub. Date: Nov. 6, 2008

(54) Tool Head for an Apparatus for the Automatic Isolation and Treatment of Cell Clones

(75) Inventor: Gerd Bornmann, Weimar (DE)

Correspondence Address: BODNER & O'ROURKE, LLP, 425 BROADHOLLOW ROAD, SUITE 108, MELVILLE, NY, 11747, US

(73) Assignee: AVISO GMBH MECHATRONIC SYSTEMS, Greiz-Gommla (DE)

(21) Appl. No.: 11/628,680

(22) PCT Filed: Jun. 4, 2005
(86) PCT No.: PCT/EP05/06021

§ 371 Date: Sep. 13, 2007

(30) Foreign Application Priority Data
Jun. 7, 2004 (DE) 10 2004 027 662.5
Sep. 25, 2004 (DE) 10 2004 046 740.4

(51) Int. Cl. C12M 1/00

(52) U.S. Cl. 435/285.1

(57) ABSTRACT

The invention relates to a tool head for a device that is used to automatically isolate and treat cell clones by means of a cloning dome, which isolates the respective cell clone from the environment, said cloning dome being held in a detachable manner by the tip of a pipette and the latter being fixed by a non-positive fit to a conical receiving element of the tool head. To hold the cloning dome on the tip of the pipette, the latter comprises a conical section, over which a complementary conical section of the cloning dome is folded and the tool head is fixed to an arm of a robot system. According to the invention, the end of the arm that is located on the tool head side is equipped with a receiving block (1), which has a lower (2) and an upper (3) stop to delimit the displacement path of a displaceable holder (4) for the pipette tip receiving element, said holder (4) being guided on the receiving block (1) by means of a linear guide (5). In addition, the upper stop (3) is configured as a graduated, laminated spring assembly (12, 13, 14) that is fixed on one side, in such a way that graduated stop forces are produced over the predetermined constant displacement path during the displacement of the arm and tool head towards a fixed point, thus producing a defined frictional connection of the conical holders of the tip of the pipette and the cloning dome.

The invention relates to a tool head for an apparatus for the automatic isolation and treatment of cell clones by means of a cloning cup for separating the respective cell clone from the environment, with the cloning cup being releasably held by a pipette tip which is fixed by frictional connection at a conical mount of the tool head, with the pipette tip further comprising a conical portion for holding the cloning cup thereon, over which a complementary conical portion at the upper part of the cloning cup is put and wherein the tool head is attached to an arm of a robotic system, in accordance with the precharacterising clause of 1.

For the cell harvest, it is possible to isolate the respective cell culture in a selective manner. For this purpose, cloning rings are employed. The cloning rings are put around the object to be harvested, and a solvent is added in a targeted manner by means of a syringe.

After a defined time interval the cell culture may then be picked up and brought to another site.

According to the solutions in the state of the art, the cloning ring has to be manually held during the entire procedure and pressed against the bottom of the vessel. Even minor movements, like slipping, result in a failure of the harvest.

In spite of the use of cloning rings, the area of the harvest is still relatively large so that the density of the cell clones on the vessel bottom has to be low in order to harvest the desired cell clones only and to avoid cross-contamination between the cultures.

In automatic apparatuses a tool head with a pick-up device is positioned above the cell culture by means of a robotic system, with the location of the culture being determined in advance for the positioning by means of optoelectronic means.

Then the tool head which is controlled by the robotic system carries out a movement in the Z direction, i.e. essentially vertically towards the cell culture and picks up the respective clone for transporting it to another site for further examination and cultivation.

Pipette tips made of synthetic material, which are filled with the solvent to be used, are employed for picking up and transporting of the cell clone to be harvested. A cloning cup is put over the pipette tip. By means of the cloning cup it is possible to isolate and thus separate the cell clone to be harvested in small areas from the environment. The addition of the solvent from the pipette tip, a subsequent rinsing operation, and the influence of the solvent causes the cell clone to be released from the bottom of the vessel, so that it can be sucked into the pipette tip. Thereafter, the above mentioned transport of the cell clone into another vessel, e.g. a titer plate, takes place for further examination.

The dedicated pipette tips with cloning cup are available under the designation “Clonetip” (registered trade mark).

It has been found that for carrying to the tool head's movement for mounting the pipette tip with cloning cup certain compressive forces for achieving a sufficient frictional connection of the respective conical couplings must be reliably ensured. These forces have, however, to be exactly maintained in such a manner that upon pulling off the cloning cup, the cup only, but not the entire pipette tip with the cell clone which is contained in the liquid therein is removed.

Based on the above, it is therefore the object of the invention to specify an advanced tool head for an apparatus for the automatic isolation and treatment of cell clones by means of a cloning cup for separating the respective cell clone from the environment, with the cloning cup being releasably held by a pipette tip which is fixed by frictional connection at a conical mount of the tool head. According to the object, the tool head is to be configured in such a manner that upon a pick-up movement carried out by a robotic system for the cloning cup and the pipette tip such forces result for the attachment of the individual parts at the tool head that upon a later pulling off of the cloning cup, the cup only, but not the pipette tip proper is removed.

The object of the invention is achieved by means of a tool head in its combination of characteristics according to the teaching of Claim 1, with the dependent claims defining at least useful embodiments and advancements.

According to the invention, a mounting block is provided for this purpose at the tool head side end of the arm which is connected with a robotic system, which comprises a lower and an upper stop for a movement in the vertical or Z direction, respectively, as a displacement travel limitation of a movable holder towards the pipette tip mount.

The holder is slidably guided at the mounting block by a linear guide.

The upper stop is formed as a staggered cantilevered plate spring group so that various stepped stop forces result over the displacement travel which is specified as being constant in the vertical or Z direction, respectively, upon a movement of the arm with tool head against a fixed point, so that a defined frictional connection of the conical holders of pipette tip and cloning cup is obtained.

Upon an attachment of the prepared Clonetip, the tip itself adheres to the pipette tip in the desired manner, on the one hand, and the mounting part of the pipette tip adheres to the complementary counterpart of the tool head, on the other hand. If during a subsequent removal of the Clonetip a tensile force is applied to same, at first the conical connection only of the Clonetip with the corresponding conical lower part of the pipette tip will be released. The latter, however, still adheres to its associated holding part of the tool head.

The pipette tip holder is formed as a hollow cylinder with a hose fitting, with the hollow cylinder at the outer circumference of the bottom end comprising a taper for mounting the pipette in a frictional connection.

Bolts for securing the plate springs are provided in the mounting block. These plate springs are cantilevered as already mentioned and attached via the bolts with the aid of sleeve parts, with the sleeve parts simultaneously serving as spacers for the plate springs.

A first lower plate spring immediately bears against the mounting block or rests on same, respectively, and comprises a single or double bend which faces the movable holder.

A second plate spring is held via a first sleeve part, with a third plate spring being supported above the second plate spring via a second sleeve part.

The plate springs may be designed as spring pairs which are arranged laterally spaced from one another.

With the first lower plate spring being subjected to a bending stress, a bend portion of it moves upwards and comes into contact with the overlying end of the second plate spring. Its bending motion is then transferred to the third overlying plate spring with the consequence that upon effecting the movement, a stepped stop force is generated which increases with an increasing travel of motion.

The hollow cylinder which serves to hold the pipette tip may additionally have recesses at the conical end which extend radially outwards, or groove-type undercuts so that a tight and secure seat of the relevant end of the pipette tip is obtained. The conical pairing of cloning cup and lower end of the pipette tip is obtained in the material combination synthetic material and synthetic material, with the conical pairing from the upper end of the pipette tip towards the conical part of the pipette tip holder to synthetic material and metal being obtained.

In the following, the invention will be explained in detail by means of an exemplary embodiment with reference to the figures, in which:

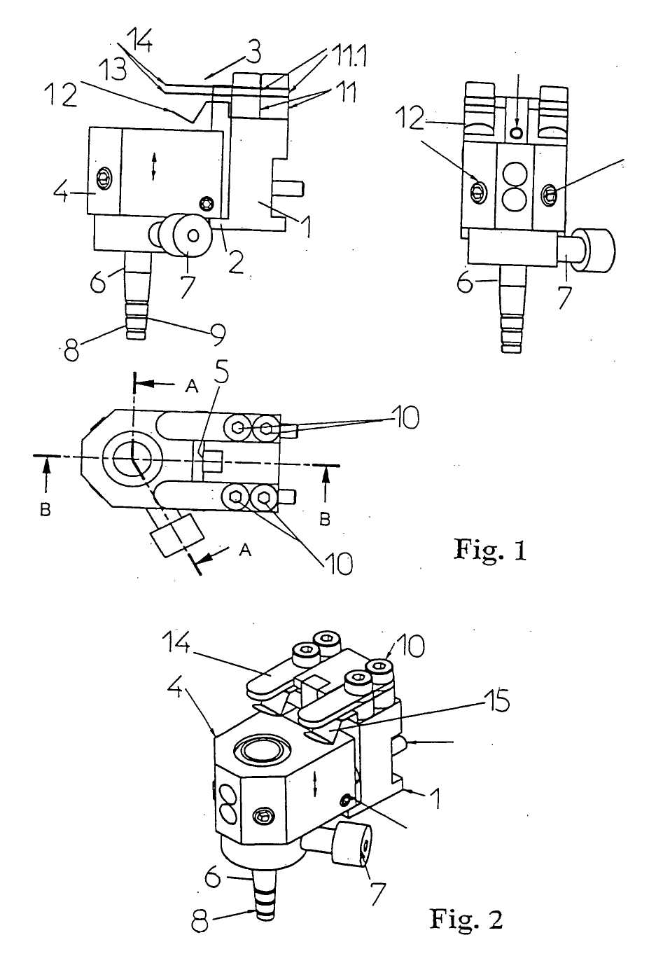

FIG. 1 is a three-way view of the tool head with plate spring stop; and

FIG. 2 is a perspective illustration of the tool head according to FIG. 1.

The tool head shown in the figures is part of an apparatus for the automatic isolation and treatment of cell clones by means of a cloning cup for the separation of the respective cell clones from the environment, with the cloning cup (now shown) being releasably held at a pipette tip (not shown) which in turn is fixed by frictional connection at a conical mount of the tool head. For holding the cloning cup at the pipette tip it comprises a conical portion over which a complementary conical portion at the upper part of the cloning cup is put.

At the tool head side end of the arm of the robotic system (not shown) a mounting block 1 is provided which comprises a lower stop 2 and an upper stop 3, which is designed in the form of a plate spring arrangement.

The upper and lower stops 2, 3 serve as displacement travel limitation of a movable holder 4 for the pipette tip mount with the movable holder 4 being guided at the mounting block 1 e.g. by a linear guide 5.

The configuration of the upper stop as staggered cantilevered plate springs results in stepped stop forces over the displacement travel which is specified as being constant upon a movement of the arm with tool head. The displacement motion of the movable holder 4 is indicated by an arrow.

The pipette tip holder proper is formed as a hollow cylinder 6 with a hose fitting 7, with the hollow cylinder at the outer circumference of the bottom end comprising a taper 8. In this conical area, radially extending grooves 9 are provided in order to improve the pipette tip's seat.

In the mounting block 1, bolts 10 are provided which mount various sleeve parts 11 as spacers for the plate springs 12, 13 and 14.

The first lower plate spring 12 is in immediate contact with the mounting block 1 and comprises a special bend, which faces the movable holder 4. This bend initially defines an upwardly directed right angle. This is followed by another right-angled bend in the opposite direction, which then extends to form a V-shaped end 15 of the plate spring 12. The tip of the V-shaped end faces the movable holder 4.

The second plate spring 13 is held via the first sleeve part 11, and the third plate spring 14 which is located above the second plate spring 13 is supported via a second sleeve part 11.1.

The plate springs may be formed as laterally spaced spring pairs, which is, in particular, best seen in the perspective view according to FIG. 2.

With the first lower plate spring 12 being subjected to a bending stress, at first a bend portion comes into contact with the overlying free end of the second plate spring 13. Its bending motion is then transferred to the third plate spring 14 which is located above.

Mounting of the pipette tip with Clonetip is effected in that the tool head is guided over a corresponding reservoir by means of the robotic system (not shown). Upon lowering, the hollow cylinder 6 with its conical end 8 comes into contact with a complementary opening end of the pipette tip. With the continuing vertical downward movement, the desired frictional connection to the conical coupling portions will occur.

Due to the special configuration of the upper stop my means of staggered plate springs it will then be ensured when pulling-off the Clonetip that the Clonetip only, but not the pipette tip with the cell clone contained therein, is removed from the holder.

1. A tool head for an apparatus for the automatic isolation and treatment of cell clones by means of a cloning cup for separating the respective cell clone from the environment, with the cloning cup being releasably held by a pipette tip which is fixed by frictional connection at a conical mount of the tool head, wherein the pipette tip further comprises a conical portion for holding the cloning cup, above which a complementary conical portion is put at the upper part of the cloning cup, and
wherein the tool head is attached to the arm of a robotic system,
characterised in that
a mounting block is provided at the tool head side end of the arm, which comprises a lower and an upper stop as a displacement travel limitation of a movable holder for the pipette tip mount, with the holder being guided at the mounting block by a linear guide,
with the upper stop being designed as a staggered cantilevered plate spring arrangement so that various stepped stop forces result over the displacement travel which is specified as being constant upon a movement of the arm with tool head against a fixed point, so that a defined frictional connection of the conical holders of pipette tip and cloning cup is obtained.

2. The tool head according to claim 1,
characterised in that
the pipette tip comprises a hollow cylinder with hose fitting, with the hollow cylinder being formed as taper at the side of the outer circumference of the bottom end.

3. The tool head according to claim 1,
characterised in that
bolts for securing the plate springs are provided in the mounting block.

4. The tool head according to claim 3,
characterised in that
the bolts mount sleeve parts which serve as spacers for the plate springs.

5. The tool head according to claim 4,
characterised in that
a first lower plate spring immediately rests on the mounting block and comprises a bend which faces the movable holder.

6. The tool head according to claim 4,
characterised in that
a second plate spring is held via a first sleeve part, with a third plate spring being supported above the second plate spring via a second sleeve part.

7. The tool head according to claim 1,
characterised in that
the plate springs are designed as laterally spaced spring pairs.

8. The tool head according to claim 5,
characterised in that
with the first lower plate spring being subjected to a bending stress, a bend portion comes into contact with the overlying free end of the second plate spring whose bending motion is then transferred to the third plate spring which is located above.

9. The tool head according to claim 2,
characterised in that
bolts for securing the plate springs are provided in the mounting block.

10. The tool head according to claim 2,
characterised in that
the plate springs are designed as laterally spaced spring pairs.

11. The tool head according to claim 3,
characterised in that
the plate springs are designed as laterally spaced spring pairs.

12. The tool head according to claim 4,
characterised in that
the plate springs are designed as laterally spaced spring pairs.

13. The tool head according to claim 5,
characterised in that
the plate springs are designed as laterally spaced spring pairs.

14. The tool head according to claim 6,
characterised in that
the plate springs are designed as laterally spaced spring pairs.

15. The tool head according to claim 6,
characterised in that
with the first lower plate spring being subjected to a bending stress, a bend portion comes into contact with the overlying free end of the second plate spring whose bending motion is then transferred to the third plate spring which is located above.
